(19) United States
(12) Patent Application Publication
(10) Pub. No.: US 2006/0264510 A1
(43) Pub. Date: Nov. 23, 2006

(54) Antiviral methods and compositions

(75) Inventors: Bruce Halstead, Grand Terrace, CA (US); Terri Halstead, Grand Terrace, CA (US)

Correspondence Address: Robert D. Fish;Suite 1400, 611 Anton Blvd., Costa Mesa, CA, 92626, US

(73) Assignee: Bio-Defense Nutritionals

(21) Appl. No.: 11/495,903

(22) Filed: Jul. 28, 2006

Related U.S. Application Data
(62) Division of application No. 10/296,061, filed on Apr. 3, 2003.

(51) Int. Cl. A61K 31/195

(52) U.S. Cl. 514/566

(57) ABSTRACT

An antiviral composition has a supply of a chelating agent that chelates an alkaline earth metal ion, wherein the chelating agent is formulated in a rectal deposition formulation, and wherein the supply of chelating agent has an immediate bioavailability. Contemplated agents when rectally administered to a subject in an effective dose in vivo promote disintegration of a virus, reduction of atherosclerotic plaques and help reducing heavy metal concentration in a subject.

### FIELD OF THE INVENTION

The field of the invention is antiviral compositions.

### BACKGROUND OF THE INVENTION

Viral infections are relatively common infectious diseases, and various methods of treating a viral infection available to a practitioner. In one method of treating a viral infection, the immune response of an immune system is stimulated. For example, in some instances the Th1 response of the patient can be increased relative to the Th2 response. An increase in the Th1 response is thought to be beneficial because many viral infections are associated with a shift in the cytokine profile toward a Th2 response, and a bias towards a Th1 response is known to be facilitated by several approaches.

In one approach, cytokines are administered to modulate the Th1/Th2 balance towards a Th1-type response. For example, Knight et al. postulate that treatment with IL-12 (Interleukine-12), a cytokine that promotes the development of Th1 cells, may be used as a treatment for AIDS since IL□12 administration has been shown to be effective at restoring cell-mediated immunity in mice infected with mouse AIDS virus or with RLV [Knight, S. C. and Patterson, S., Annu. Rev. Immunol. 1994. 15: 593-615]. In another example, Gracie, J. A. et al., demonstrated that administration of IL-18 to mice exhibited pleiotropic activities critical to the development of Th1 responses. [Gracie et al. J Clin Invest 1999 Nov 15; 104(10):1393-1401]. Although the administration of cytokines typically results in relatively specific increases in desired Th1 cytokines, prolonged administration of cytokines may be problematic for various reasons. For example, the production of recombinant cytokines is relatively expensive, and isolation of non-recombinant cytokines from natural sources is generally difficult due to the very low concentration of cytokines in natural sources. Moreover, depending on the nature of the cytokine, cytokines may not be well tolerated in patients.

In another approach, immunomodulatory substances other than cytokines may be employed to increase the Th1 response. For example, Sprietsma J. E. suggests that zinc ions (Zn²⁺) and nitric oxide (NO), together with glutathione (GSH) and its oxidized form, GSSG, may help to regulate an immune response to antigens [Sprietsma J. E; Med Hypotheses 1999 July; 53(1):6-16]. The author reports in more detail that deficiencies of Zn²⁺, NO and/or GSH shift the Th1/Th2 balance towards Th2, and that replenishment with Zn²⁺, NO and/or GSH may shift the Th1/Th2 balance towards Th1. Administration of Zn²⁺ or GSH/GSSG is especially advantageous, since these substances are non-toxic at even elevated concentrations, and inexpensive to produce. Furthermore, Zn²⁺ and GSH/GSSG preparations may be orally administered, and therefore significantly reduce the risk of allergic reactions, especially when the preparations are not ultrapure. However, the administration of Zn²⁺ and/or GSH/GSSG seems to be beneficial only to restore a Th1/Th2 balance from a Th2 dominated state, whereas it is unclear if administration of Zn²⁺ and/or GSH/GSSG may increase a Th1 response from a normal Th1/Th2 balance.

In another method of treating a viral infection, the virus is directly targeted with an appropriate anti-viral drug. For example, patients infected with the HIV virus often receive a cocktail of drugs to block virus propagation, and various classes for direct anti-viral treatment are known in the art. Some direct anti-viral drugs block the reverse transcriptase of a retrovirus. Reverse transcriptase (RT) inhibitors are typically nucleoside analogs such as AZT, 3TC, or ddI. Alternatively, non-nucleoside RT inhibitors, including quercetin may be employed. In vitro, RT inhibitors are typically potent anti-viral drugs. However, in vivo, and especially during a period of relatively fast viral replication, the generation of RT inhibitor resistant virus mutants is problematic. Moreover, many RT inhibitors also exhibit undesirable activity on DNA replication in the host organism and significant cytotoxicity at elevated concentrations, thereby limiting the concentration that may be administered without severe side effects.

Among other direct anti-viral drugs are the protease inhibitors, which block or interfere with virus protein processing. Protease inhibitors are typically highly specific towards the viruses' proteolytic enzymes, however, due to their mostly hydrophobic nature, administration at desirable concentrations often becomes problematic. Another problem is that development of cross-resistance and severe side effects frequently occur. In order to reduce the development of multidrug resistant virus strains, mixtures of RT inhibitors and protease inhibitors may be prescribed. Although such mixtures are presently employed relatively successfully, the relatively high occurrence of adverse side effects and the potential of generating multidrug resistant virus strains persist.

To circumvent at least some of the problems associated with side effects and relatively high costs of antiviral drugs, Bennett et al. describe in U.S. Pat. No. 5,602,180 the use of EDTA complexes in a suppository. The use of chelating agents, including EDTA, has been found to promote disintegration of retroviruses [Wunderlich, V. and Sydow, G. Arch. Virol. 1982, 73:171-183]. Bennett's suppositories contain disodium EDTA and controlled-release agents, which release the disodium EDTA over a period of about three to four hours after rectal placement of the suppository. However, Bennett's suppositories are limited to disodium EDTA that exhibits relatively moderate selectivity between Mg²⁺ and Ca²⁺.

Although various antiviral compositions and antiviral treatments are known in the art, all or almost all of them have one or more disadvantages. Therefore, there is a need to provide improved methods and compositions for treatment of viral infections.

### SUMMARY OF THE INVENTION

The present invention is directed to an antiviral composition having a supply of chelating agent that chelates an alkaline earth metal ion, wherein the chelating agent is formulated in a rectal deposition formulation, and wherein the supply of chelating agent has an immediate bioavailability. When rectally administered to a subject in an effective dose in vivo, contemplated agents promote disintegration of a virus.

In one aspect of the inventive subject matter, generally preferred chelating agents are various chelators other than ethylenediamine-N,N,N′,N′-tetraacetic acid (EDTA), chelate Ca²⁺ and/or Mg²⁺, and include at least three carboxylic acid groups. While particularly preferred chelating agents include at least three acetic acid groups, especially contemplated chelating agents are 1,2-Bis(2-aminophenoxy)ethane-N,N,N′,N′-tetraacetic acid (BAPTA), Ethylenebis(oxyethylenenitrilo)tetraacetic acid (EGTA), 1,2-bis(2-aminophenoxy)ethane-N,N,N′,N′-tetraacetic acid tetrakis(acetoxymethyl ester) (BAPTA-AM), diethylenetriamine-pentaacetic acid (DTPA), trimethylaminetricarboxylic acid (NTA), trans-1,2-diaminocyclohexane-tetraacetic acid (CDTA), poly(aspartic acid), and poly(glutamic acid).

In another aspect of the inventive subject matter, contemplated viruses include a retrovirus, and especially contemplated retroviruses include the HIV virus. Preferred rectal deposition formulations are a liquid or a solid, and where the rectal deposition formulation is a solid and administered to the colon of a subject, substantially of the supply of chelating agent is present in the colon in a readily absorbable form in less than 2 hours, preferably less than 1 hour, and more preferably less than 30 minutes. With respect to the effective dose in a rectal administration, it is contemplated that the chelating agent is employed in an amount of 500 mg, and more preferably 1500 mg.

In yet another aspect of the inventive subject matter, chelating agents other than EDTA may also be employed for purposes other than antiviral treatment, including heavy metal detoxification, and reduction of atherosclerotic plaques, wherein the chelating agent may be orally or parenterally administered.

Various objects, features, aspects and advantages of the present invention will become more apparent from the following detailed description of preferred embodiments of the invention.

### DETAILED DESCRIPTION

As used herein, the term “chelating agent” refers to a molecule that binds a metal ion and/or an alkaline earth metal ion via a non-covalent bond, most commonly a coordinate bond, with a K_{D} of less than 10⁻³ mol⁻¹, wherein the chelating agent may be in acid form, base form or a salt form. For example, EGTA in protonated or sodium salt form is considered a free chelating agent, because EGTA binds Mg²⁺ and Ca²⁺ with a K_{D} of less than 10⁻³ mol⁻¹.

As also used herein, the term “immediate bioavailability” means that a composition or molecule is present in an active form in a formulation such that a substantial portion of a dose of the composition or molecule exhibits some systemic chelating effect within minutes, preferably within less than 15 min, more preferably within less than 10 min, and most preferably within less than 5 min. For example, a molecule that is dissolved in a carrier solution is regarded to have immediate bioavailability.

It is known that retroviruses can be disintegrated by chelating agents, especially by agents that chelate Mg²⁺ and/or Ca²⁺, and that chelating agents may further reduce infectivity of certain viruses [Wunderlich, V. and Sydow, G. Arch. Virol. 1982, 73:171-183]. Thus, it is contemplated that an antiviral composition generally has a supply of a chelating agent that chelates an alkaline earth metal ion, and it is particularly contemplated that the chelating agent in the antiviral composition is formulated in a rectal deposition formulation, wherein the supply of chelating agent has an immediate bioavailability.

It should be appreciated that many chelating agents are known in the art, and that all of the known chelating agents are contemplated for use herein. It is generally preferred that contemplated chelating agents include at least three carboxylic acid groups, all of which are preferably acetic acid groups. Although not excluded, it is further contemplated that appropriate chelating agents are chelating agents other than EDTA. The choice of the particular chelating agent is predominantly determined by the desired physicochemical properties and tolerability of the chelating agent. For example, where a relatively high solubility (e.g., 1M) is desired, EGTA, CDTA or NTA may advantageously be employed. Where a more pronounced selectivity of chelation towards Ca²⁺ is desirable, BAPTA may be utilized, and BAPTA-AM may be particularly suitable where sequestration of Ca²⁺ within a cell is desired. Alternatively, contemplated chelating agents may include DTPA, NTA, and polymeric forms of aspartic acid, glutamic acid, and any reasonable combination thereof.

With respect to viruses that can be disintegrated and/or reduced in infectivity, virus particles that require Ca²⁺ and/or Mg²⁺ for structural integrity of their envelope are generally contemplated and include DNA and RNA viruses. Particularly contemplated RNA viruses are retroviruses in general, and HIV in particular. Further especially contemplated viruses include the hepatitis C and hepatitis D virus. Contemplated DNA viruses include polyomaviruses, HBV, etc. However, many more viruses are also contemplated, and a collection of appropriate viruses are listed in Fields Virology, Third Edition (Lippincot Williams & Wilkins), pages 40-41, 52, and 1767-1847, and Arch. Virol. 1982, 73:171-183, both of which are incorporated by reference herein.

It is still further contemplated that chelating agents are preferably formulated in a rectal deposition formulation, which may be in solid or liquid form. Where the formulation is in a solid form, it is further contemplated that appropriate forms include dissolvable carriers such as wages, fatty acids and oils with melting points of about 30°-35° C. Especially preferred formulations are formulations known in the art that are employed in the fabrication of rectal suppositories, so long as such formulations allow an immediate bioavailability. Thus, where a supply of chelating agent is administered into the colon of a subject in a solid form, it is particularly contemplated that substantially all of the supply of chelating agent is present in the colon in a readily absorbable form in less than 2 hours, more preferably less than 1 hour, and most preferably less 30 minutes after the administration of the formulation. Availability of the chelating agent or a portion of the chelating agent in less that 2 hrs, less than 1 hr or less than 30 min may be achieved by a variety of time release formulations, and contemplated time release formulations may include formulations with a melting point of less than 37° C., enzymatically degradable carriers, dissolving or swellable carriers, etc. Thus, it is contemplated that an entire dose of chelating agent may be available (or released from the time release formulation) in less than 2 hours, preferably less than 1 hour, and even more preferably in less than 30 min. A particular advantage of such time release formulations is that relatively high dosages may be administered that might otherwise pose a potential risk if administered without a time release formulation. However, it should be appreciated that administrations without time release may safely be administered by employing smaller dosages at multiple administrations.

Where the formulation is in a liquid form, it is contemplated that appropriate liquid forms may include buffered and unbuffered solutions, solutions with relatively high viscosity such as gels, creams, foams and ointments, which may or may not have a decreased viscosity at elevated temperatures. Liquid forms are particularly advantageous, since the delivery of the chelating agent is almost instantaneous. Where the solutions are buffered, it is contemplated that the buffers have an alkaline pH, and a preferred pH range is a range between 8.0 and 10.0.

Alternatively, the chelating agent may also be administered in various alternative routes, and it is especially contemplated that where the chelating agent is an agent other than EDTA that appropriate routes include oral and parenteral administration. For example, CDTA may be orally administered in form of an acid resistant caplet or capsule. However, oral administration need not be limited to a caplet or capsule, and alternative oral administrations include syrups, powders, tablets, etc. In another example, EGTA may be parenterally administered by intravenous injection. It is contemplated, however, that alternative parenteral administrations may also include inhalation, transdermal delivery, injections into sites other than a vein, etc.

In a particularly contemplated aspect of the inventive subject matter, it is preferred that the administration of the chelating agent is accompanied by (preferably oral) administration of a nutritional supplement. Preferred nutritional supplements include supplements that help replenish calcium levels and particularly preferred supplements include aragonite calcium carbonate from fossil coral minerals. Other contemplated supplements that include herbal products (e.g., adaptogenic formulations with no apparent cytotoxicity) are contemplated to assist in inhibition of viral replication (e.g., by inhibiting the production of reverse transcriptase). It is further contemplated that such supplements may also help boost the immune system and potentially improve overall vitality and stamina. It is further contemplated that such adaptogenic supplements are considered to have tumor preventive and radio-protective properties, and may help increase the functioning of the immune system by increasing the T-cell population. Exemplary compositions for contemplated nutritional supplements are shown in Tables 1 and 2.

TABLE 1

| Ingredient | Amount (mg/tablet) |
|---|---|
| Arcticum lappa | 40 mg (10:1 concentrate) |
| Viola yedoensis | 40 mg (10:1 concentrate) |
| Andrographis paniculata | 40 mg (10:1 concentrate) |
| Lonicera erythrorhizon | 40 mg (10:1 concentrate) |
| Epimedium saggittatum | 40 mg (10:1 concentrate) |

TABLE 2

| Ingredient | Amount (mg/tablet) |
|---|---|
| Arcticum lappa | 10 mg (10:1 concentrate) |
| Viola yedoensis | 10 mg (10:1 concentrate) |
| Andrographis paniculata | 10 mg (10:1 concentrate) |
| Lonicera erythrorhizon | 10 mg (10:1 concentrate) |
| Altemanthera philoeroides | 10 mg (10:1 concentrate) |

It should be appreciated, however, that various additional ingredients may be added to the supplement depicted in Table 1 and 2 to either enhance or modulate the activity of the herbal components.

With respect to the amount of chelating agent it is contemplated, that the chelating agent is administered to a subject in vivo in a dose effective to promote disintegration of a virus in the subject. The actual dose of the chelating agent may thereby vary among individual subject and may further be determined by the particular virus that is to be disintegrated. Therefore, an effective dose may comprises rectal administration of the chelating agent between about 5 mg-2500 mg, and generally contemplated doses include rectal administration of 500 mg or 1500 mg of the chelating agent. However, where even higher dosages of the chelating agent are required, or where it is preferred to maintain relatively high dosages over an extended period of time, multiple dosages are also contemplated.

It should further be appreciated that appropriate formulations may further comprise active and/or inactive ingredients. For example, active ingredients may include compositions to stimulate the immune system, an immunomodulating composition, a coral mineral product, compositions to facilitate uptake of the chelating agent into the blood stream, or direct antiviral compounds such as nucleoside analogs, etc. The term “immunomodulating composition” as used herein refers to a composition that enhances at least one of a humoral and cellular response towards a challenge. For example, an immunomodulating composition may increase an antibody titer against a challenge, or an activity of cytotoxic T-lymphocytes. Inactive ingredients may include fillers, coloring agents, thixotropic compositions, and foam building agents.

In an exemplary use, a person diagnosed with an HIV infection receives twice daily an enema of 20 ml of a 50 mg/ml solution of EGTA in 10 mM sodium phosphate buffer pH8.4 for at least 30 consecutive days. It should be recognized, however, that the exemplary use need not be limited to the specified amounts and times, but treatment schedules may vary considerably. For example, where the person already receives an antiviral medication (e.g., protease inhibitor cocktail, RT-inhibitor, etc.), lower dosages or less frequent administrations are contemplated, while in cases where the person does not receive another antiviral treatment, higher dosages and more frequent administrations are contemplated. It is also contemplated that the antiviral composition may be employed in a preventative fashion, i.e., the antiviral composition may be employed in a person that is not infected with a virus.

It is still further contemplated that the compositions according to the inventive subject matter may have advantageous properties and uses in therapeutic applications other than antiviral activity, especially where the chelating agent is a substance other than EDTA, and particularly contemplated uses include heavy metal detoxification in animal and human, and reduction of atherosclerotic plaque.

With respect to heavy metal detoxification in animal and human, it is known in the art that upon oral administration or injection EDTA complexes various metals and heavy metals other than Ca²⁺, and oral administration or injection of EDTA has therefore found widespread use in detoxifycation of some heavy metal poisonings. Various alternative oral or injectable chelation agents for heavy metals have also been described [e.g., Llobet, J. M. et al. Arch. Environ. Contam. Toxicol. 1990, 19(2): 185-9; Treatment of acute lead intoxication. A quantitative comparison of a number of chelating agents. Llobet, J. M. et al. Arch. Toxicol. 1988, 61(4):321-3; Antidotes for zinc intoxication in mice] and include oral and injectable forms of penicillamine, 2,3-dimercaptosuccinic acid, and 2,3-dimercapto-1-propanesulfonate. However, it is not known to the inventors that chelators other than EDTA have been used for detoxification of heavy metals in animal and human via rectal administration. Rectal administration is particularly advantageous for various reasons. For example, suppositories can be self-administered by almost all patients. Furthermore, rectal administration inflicts only relatively low discomfort to the patient. Moreover, rectal administration bypasses the stomach, a highly acidic environment that may lead to at least partial destruction of some of the chelating agents.

Therefore, it is contemplated that rectal administration of chelating agents may also be employed in a method to reduce a heavy metal concentration in a subject, wherein in one step a chelating agent is provided that chelates a metal ion, wherein the chelating agent is formulated in a rectal deposition formulation and wherein the supply of chelating agent has an immediate bioavailability. Alternatively, the rectal deposition formulation may further comprise a time release agent to release the chelating agent in a period of between 0-30 min, 30-60 min, 60-120 min, 120-180 min, or longer. In another step, the chelating agent is rectally administered to the subject in a concentration effective to reduce the heavy metal ion concentration.

It is generally contemplated that the heavy metal may be in elemental or ionic form, and particularly contemplated heavy metals include mercury, Zn²⁺, Cu⁺, Cd²⁺, and Co²⁺. However, various alternative metals and their ionic forms are also contemplated, including nickel, arsenic, selenium, iron, mercury, chromium, antimony, beryllium, thallium, silver, scandium, titanium, vanadium, chromium, manganese, etc. While it is generally contemplated that all known chelating agents may be suitable for reduction of heavy metals in a subject, it is particularly preferred that the chelating agent comprises a plurality of carboxylic acid groups and it is even more preferred that the chelating agent is EDTA, EGTA, CDTA, or DTPA. With respect to the rectal deposition formulation the same considerations as already described above apply.

An exemplary method of reducing a heavy metal concentration in a subject may therefore comprise a single rectal administration of 20 ml of a 10 mg/ml buffered aqueous solution of CDTA three times daily over a period of about 15-20 days. It should be recognized, however, that depending on the particular heavy metal, the site of accumulation, and the concentration of the heavy metal in the subject many treatment schedules other than a single rectal administration of 20 ml of a 10 mg/ml buffered aqueous solution of CDTA three times daily over a period of about 15-20 days are also appropriate.

For example, where treatment is prophylactic or necessitated by relatively low concentrations of a heavy metal, total daily dosages of less than 600 mg are contemplated, including total daily dosages of 200-600 mg, 50-200 mg, and less that 50 mg. Likewise, where acute and/or severe heavy metal intoxications are to be treated by a method according to the inventive subject matter, higher total daily dosages of more than 600 mg are contemplated, including total daily dosages of 600-1500 mg, 1500-2500 mg, and more than 2500 mg. With respect to the formulation it should be appreciated that numerous alternative formulations are also appropriate, and contemplated alternative formulations include the formulations already described above. Similarly, it should be appreciated that various alternative administration periods other than a period of about 15-20 days are also appropriate, including single administrations in cases where treatment is prophylactic, or administration over a period of less than 15 days, where the heavy metal concentration is relatively low. On the other hand, where the heavy metal is predominantly is tissues that bind the heavy metal relatively firmly (e.g. lipophilic tissue) administrations of 2-6 weeks and longer are contemplated.

With respect atherosclerotic plaques it is contemplated that rectal administration of chelating agents may also be employed in a method to reduce a atherosclerotic plaques in a subject, wherein in one step a chelating agent is provided that chelates an alkaline earth metal ion, wherein the chelating agent is formulated in a rectal deposition formulation and wherein the supply of chelating agent has an immediate bioavailability. In another step, the chelating agent is rectally administered to the subject in a concentration effective to reduce the atherosclerotic plaque in a subject. As used herein, the term “reducing the atherosclerotic plaque” refers to a gross reduction in size and/or volume of one or more atherosclerotic plaques, which may also include complete disappearance of the atherosclerotic plaque or plaques.

In an exemplary method of reducing atherosclerotic plaque, a person diagnosed with atherosclerotic plaques receives once daily an enema of 10 ml of a 50 mg/ml solution of EGTA in 10 mM sodium phosphate buffer pH8.4 for a period of about 12 weeks. However, it should be appreciated that the exemplary method need not be limited to the specified amounts and times, and formulation and treatment schedules may vary considerably. For example, where the person already underwent a vasodilation procedure, lower dosages or less frequent administrations are contemplated, while in cases where the person did not receive previous treatment to reduce the atherosclerotic plaques, higher dosages and more frequent administrations are contemplated.

Likewise, the chelating agent need not be limited to EGTA, but may be various alternative chelating agents including EDTA, CDTA, and DTPA, wherein the choice of the chelating agent will predominantly depend on the desired specificity of the chelator and the tolerability at a particular concentration. Furthermore, the formulation of the chelating agent need not be restricted to 10 ml of a 50 mg/ml solution of EGTA in 10 mM sodium phosphate buffer pH8.4. For example, alternative formulations may be employed to achieve a larger distribution, faster absorbption, etc., and appropriate formulations include those already described above.

Thus, specific embodiments and applications of antiviral compositions have been disclosed. It should be apparent, however, to those skilled in the art that many more modifications besides those already described are possible without departing from the inventive concepts herein. For example, the route of administration need not necessarily be restricted to a rectal administration of the chelating agent, but may also include vaginal administration. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1-37. (canceled)

38. A method of providing an antiviral composition, comprising:
providing a supply of chelating agent that chelates an alkaline earth metal ion; and
dissolving or dispersing the chelating agent in a liquid rectal deposition formulation to thereby form the antiviral composition;
wherein the chelating agent is dissolved or dispersed in the liquid rectal deposition formulation such that the entire supply of the chelating agent is released into the colon in a period of less than 30 minutes.

39. The method of claim 38 wherein the chelating agent, when administered to a subject in an effective dose in vivo, promotes disintegration of a virus.

40. The method of claim 39 wherein the effective dose comprises rectal administration of 500 mg of the chelating agent.

41. The method of claim 39 wherein the effective dose comprises rectal administration of 1500 mg of the chelating agent.

42. The method of claim 38 wherein the chelating agent comprises at least three carboxylic acid groups.

43. The method of claim 38 wherein the chelating agent is selected from the group consisting of 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid.

44. The method of claim 38 wherein the chelating agent is selected from the group consisting of trimethylaminetricarboxylic acid, poly(aspartic acid), and poly(glutamic acid).

45. The method of claim 38 wherein the chelating agent is a chelator other than ethylenediamine-N,N,N′,N′-tetraacetic acid.

46. The method of claim 38 wherein the chelating agent is present at a concentration effective to have antiretroviral activity.

47. The method of claim 46, wherein the chelating agent is present at a concentration effective to exhibit antiretroviral activity against an HIV virus.

48. The method of claim 38 wherein the chelating agent is present at a concentration effective to have antiviral activity against at least one of a hepatitis C virus and a hepatitis D virus.

49. The method of claim 39 wherein, when administered to the colon of a subject, substantially all of the supply of chelating agent is immediately present in the colon in a readily absorbable form.

50. The method of claim 40 wherein, when administered to the colon of a subject, substantially all of the supply of chelating agent is immediately present in the colon in a readily absorbable form.

51. The method of claim 41 wherein, when administered to the colon of a subject, substantially all of the supply of chelating agent is immediately present in the colon in a readily absorbable form.

52. The method of claim 46 wherein, when administered to the colon of a subject, substantially all of the supply of chelating agent is immediately present in the colon in a readily absorbable form.

53. The method of claim 38 further comprising a step of providing a separate active ingredient formulated for oral administration.

54. The method of claim 53 wherein the separate active ingredient is selected from the group consisting of aragonite calcium carbonate from coral and an immunomodulating composition.

55. The method of claim 54 wherein the active ingredient comprises a plant extract combination that includes a plant extract selected from the group consisting of Arctium lappa extract, Viola yedoensis extract, Andrographis paniculata extract, Lonicera erythrorhizon extract, Epimedium saggittatum extract, and Altemanthera philoxeroides extract.

56. The method of claim 55 wherein the plant extract combination comprises 40 mg of a 10:1 concentrate of each of a Arcticum lappa extract, Viola yedoensis extract, Andrographis paniculata extract, Lonicera erythrorhizon extract, and Epimedium saggittatum extract.

57. The method of claim 55 wherein the plant extract combination comprises 40 mg of a 10:1 concentrate of each of a Arcticum lappa extract, Viola yedoensis extract, Andrographis paniculata extract, Lonicera erythrorhizon extract, and Altemanthera philoeroides extract.
